(19) United States
(12) Patent Application Publication
(10) Pub. No.: US 2020/0108064 A1
(43) Pub. Date: Apr. 9, 2020

(54) LOW-DOSE BRIMONIDINE COMBINATIONS AND USES THEREOF

(71) Applicant: Eye Therapies, LLC, Dana Point, CA (US)

(72) Inventor: Gerald Horn, Deerfield, IL (US)

(21) Appl. No.: 16/701,803

(22) Filed: Dec. 3, 2019

Related U.S. Application Data
(63) Continuation-in-part of application No. 16/257,710, filed on Jan. 25, 2019.
Application No. 16/257,710 is a continuation-in-part of application No. 16/002,106, filed on Jun. 7, 2018.
(60) Provisional application No. 62/621,082, filed on Jan. 24, 2018.
(60) Provisional application No. 62/516,931, filed on Jun. 8, 2017.

(51) Int. Cl. A61K 31/498; A61K 45/06; A61K 9/00; A61K 31/472; A61P 27/06

(52) U.S. Cl. CPC A61K 31/498; A61K 45/06; A61P 27/06; A61K 31/472; A61K 9/0048

(57) ABSTRACT

The present invention is related to compositions containing an ophthalmological drug and low-dose brimonidine. The present invention is further related to methods of treating ophthalmological disease by administering compositions of the present invention. The present invention is further related to methods of increasing residency time of ophthalmological drugs on the surface of an eye by co-administering low-dose brimonidine.

### FIELD OF THE INVENTION

The present invention is related to compositions containing an ophthalmological drug and low-dose brimonidine. The present invention is further related to methods of treating ophthalmological disease by administering compositions of the present invention. The present invention is further related to methods of increasing residency time of ophthalmological drugs on the surface of an eye by co-administering low-dose brimonidine.

### BACKGROUND OF THE INVENTION

Ophthalmological drugs are used to treat various diseases such as glaucoma, allergies, macular degeneration, uveitis and others. A major issue with the treatment of many eye diseases is patient compliance. Patients with eye diseases must instill medications on a consistent basis to achieve sustained relief. The frequency of administration for which the patient must adhere is tedious and often leads to a lack of compliance.

Further, topical application of ophthalmological drugs is complicated by residency time on the surface of the eye and system absorption of the drug. Ophthalmological drugs are often removed readily from the eye due to the natural turnover of the tear film. Further, ophthalmological drugs are also systemically absorbed through the vasculature of the eye. The amount of drug that must be applied to the eye due to tear film turnover combined with the systemic absorption of that drug generally leads to systemic side effects, which exacerbates the lack of patient compliance.

Brimonidine, a known alpha-2 (a-2) adrenergic receptor agonist, is currently available under the tradenames Alphagan® (Alphagan is a registered trademark of Allergan, Inc.) and Lumify® (Lumify is a registered trademark of Bausch and Lomb, Inc.) and in the form of 0.1%, 1.5% and 0.2% brimonidine tartrate. Brimonidine has been shown to induce vasoconstriction of the capillaries in the eye at low doses. This vasoconstriction leads to reduced redness and whitening of the sclera. See, U.S. Pat. No. 9,259,425; U.S. Pat. No. 8,987,270; U.S. Pat. No. 8,765,758; U.S. Pat. No. 8,580,787; and U.S. Pat. No. 8,293,742. However, these benefits appear to be mostly cosmetic.

Thus, there is a further need in the art for a composition and treatment that will lead to less side effects and greater patient compliance when using ophthalmological drugs. Ideally, this treatment will not result in further side effects.

### SUMMARY OF THE INVENTION

In one embodiment, the present invention is directed to an ophthalmological composition for the treatment of an eye disease comprising an ophthalmological drug and brimonidine at a concentration from about 0.005% to about 0.10% w/v.

In another embodiment, the present invention is directed to a method of treating an eye disease comprising administering a composition of the present invention.

In another embodiment, the present invention is directed to a method of treating an eye disease comprising topically administering a composition comprising an ophthalmological drug concurrently or sequentially with brimonidine at a concentration from about 0.005% to about 0.10% w/v to a subject in need thereof.

In another embodiment, the present invention is directed to a method of increasing ophthalmological drug residency time on the surface of an eye comprising topically administering the ophthalmological drug concurrently or sequentially with brimonidine at a concentration from about 0.005% to about 0.10% w/v to a subject in need thereof

In another embodiment, the present invention is directed to a method of treating an eye disease, wherein the method further provides eye redness reduction.

In another embodiment, the present invention is directed to a method of treating an eye disease, wherein the method further provides eye whitening.

In another embodiment, the present invention is directed to a method of treating an eye disease, wherein the method further provides conjunctival swelling reduction as well as hyperemia reduction and eye whitening.

### BRIEF DESCRIPTION OF THE DRAWINGS

FIG. 1 demonstrates a prophetic example of IOP reduction after administration of 0.005% latanoprost (0 hours, white bar), versus IOP reduction after administration of 0.03% brimonidine and 0.005% latanoprost (4, 6 and 8 hours, black bar).

### DETAILED DESCRIPTION OF THE INVENTION

It is a discovery of the present invention that postcapillary venular leakage may be reduced selectively by low dose brimonidine over alpha 1 vasoconstrictors. This selective vasoconstriction reduces the systemic absorption of ophthalmological drugs thus increasing the residency time of these drugs on the eye. This increased residency time lead to greater effectiveness of the ophthalmological drug allowing a reduction in the amount of drug instilled per administration and/or the frequency of administration. This reduction in amount and/or frequency of administration should lead to less side effects and greater patient compliance.

In one embodiment, the present invention is directed to an ophthalmological composition for the treatment of an eye disease comprising an ophthalmological drug and brimonidine at a concentration from about 0.005% to about 0.059% w/v.

In another embodiment, the present invention is directed to a method of treating an eye disease comprising administering a composition of the present invention.

In another embodiment, the present invention is directed to a method of treating an eye disease comprising topically administering a composition comprising an ophthalmological drug concurrently or sequentially with brimonidine at a concentration from about 0.005% to about 0.10% w/v to a subject in need thereof.

In another embodiment, the present invention is directed to a method of increasing ophthalmological drug residency time on the surface of an eye comprising topically administering the ophthalmological drug concurrently or sequentially with brimonidine at a concentration from about 0.005% to about 0.10% w/v to a subject in need thereof

The term “brimonidine” encompasses, without limitation, brimonidine salts and other derivatives, and specifically includes, but is not limited to, brimonidine tartrate, 5-bromo-6-(2-imidazolin-2-ylamino)quinoxaline D-tartrate, Alphagan® (Alphagan is a registered trademark of Allergan, Inc.), and UK14,304.

Brimonidine may be present in compositions or methods of the present invention at a concentration from about 0.005% to about 0.10% w/v, preferably from about 0.01% to about 0.075% w/v, preferably from about 0.005% to about 0.059% w/v, preferably from about 0.005% to about 0.050% w/v, preferably from about 0.005% to about 0.050% w/v, preferably from about 0.01% to about 0.059% w/v preferably from about 0.01% to about 0.050% w/v, preferably from about 0.025% to about 0.045% w/v, preferably from about 0.06% to about 0.1% w/v, preferably from about 0.06% to about 0.07% w/v, preferably from about 0.035% to about 0.050% w/v, preferably from about 0.025% to about 0.035% w/v and preferably about 0.025%, about 0.035%, about 0.04%, about 0.05% w/v about 0.065% w/v or about 0.073% w/v.

The term “glaucoma drug” or “second glaucoma drug” encompasses, without limitation, all drugs used to treat glaucoma including those drugs used to lower intraocular pressure with the exception of brimonidine.

The term “histamine antagonist” includes all chemicals that have been found to antagonize at least one histamine receptor including histamine H₁ and histamine H₂.

The term “allergy” or “allergies” or “allergic response” refers to allergic rhinitis and or ocular allergies. Allergic rhinitis is an allergic inflammation of the nasal airways with attendant symptoms, including, but not limited to, rhinorrhea, nasal obstruction, nasal itching, sneezing, ocular pruritis. Ocular allergies are any allergic diseases of the eye, including, but not limited to, seasonal/perennial allergic conjunctivitis, vernal keratoconjunctivitis, giant papillary conjunctivitis, perennial allergic conjunctivitis and atopic keratoconjunctivitis.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either net or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either net or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isbutyric, oxalic, maleic, malonic, benzoic, succinic, suberic, fumeric mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present inventions contain both basic and acidic functionalities that allow the compounds to be converted into either base or acid addition salts.

The neutral forms of the compounds may be registered by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In additional to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are often useful because, in some situations, they may be easier to administer than the parent drug. They may, be bioavailable by oral administration whereas the parent drug is not. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present invention which is administered as an ester (the “prodrug”), but then is metabolically hydrolyzed to the carboxylic acid, the active entity. Additional examples include peptidyl derivatives of a compound of the invention.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

In preferred embodiments, compositions of the present invention may comprise a nonionic surfactant, optionally, a viscosity enhancer, preferably hydroxypropylmethyl cellulose (“HPMC”) or carboxymethyl cellulose (“CMC”), a buffer, optionally, sorbate, optionally, a polyol, preferably mannitol, optionally, a salt, preferably sodium chloride and optionally, a preservative, preferably EDTA.

Nonionic surfactants suitable for use in the present invention include, but are not limited to a polysorbate, a poloxamer, a polyoxyl, an alkyl aryl poly ether, a cyclodextrin, a tocopheryl, polyethylene glycol succinate. a glucosyl dialkyl ethers and a crown ether, ester-linked surfactants. Nonionic surfactants may be present in compositions of the present invention at a concentration from about 1% to about 5% w/v.

Polysorbates suitable for use in the present invention include, but are not limited to, polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate) and polysorbate 80 (polyoxyethylene (20) sorbitan monooleate.

Polysorbates may be present in compositions of the present invention at a concentration from about 1% to about 5% w/v, more preferably from about 2% to about 4% w/v and most preferably at about 2.5% or about 4.0% w/v.

Cyclodextrins suitable for use in the present invention include, but are not limited to, ionically charged (e.g. anionic) beta—cyclodextrins with or without a butyrated salt (Captisol) 2-hydroxypropyl beta cyclodextrin (“HPβCD”), alpha cyclodextrins and gamma cyclodextrins.

Poloxamers include but are not limited to poloxamer 103, poloxamer 123, and poloxamer 124, poloxamer 407, poloxamer 188, poloxamer 338 and any poloxamer analogue or derivative

Polyoxyls include but are not limited to Brij® 35, 78, 98, 700 (polyoxyethylene glycol alkyl ethers) and Spans (sorbitan esters) and Span® 20-80 (sorbitan monolaurate, sorbitan monopalmitate, sorbitan monostearate, and sorbitan monooleate).

As used herein the term “polyol” refers to compounds with multiple hydroxyl functional groups available for organic reactions such as monomeric polyols such as glycerin, pentaerythritol, ethylene glycol and sucrose. Further, polyols may refer to polymeric polyols including glycerin, pentaerythritol, ethylene glycol and sucrose reacted with propylene oxide or ethylene oxide. In a preferred embodiment, polyols are selected from the group consisting of mannitol, glycerol, erythritol, lactitol, xylitol, sorbitol, isosorbide, ethylene glycol, propylene glycol, maltitol, threitol, arabitol and ribitol. In a more preferred embodiment, the polyol is mannitol. Polyols may be present in composition of the present invention at a concentration from about 0.1% to about 5% w/v, more preferably from about 0.5% to about 2.5% w/v and most preferably at about 0.75% w/v.

Viscosity enhancers suitable for use in the present invention include, but are not limited to, cellulose derivatives, carbomers, gums and hyaluronates. In a preferred embodiment the cellulose derivative is HPMC or CMC at a concentration from about 0.1% to about 1.75% w/v, preferably at about 1.2% w/v. In another preferred embodiment the cellulose derivative is CMC at a concentration from about 0.75% to about 1.75% w/v, preferably 1.4% w/v.

Preservatives suitable for use in the present invention include, but are not limited to, benzalkonium chloride (“BAK”), sorbate, EDTA, methyl paraben or peroxide based preservatives.

Antioxidants suitable for use in the present invention include, but are not limited to, citrate, EDTA, sodium metabisulfite, sodium thiosulfate, acetylcysteine, butylated hydroxyanisole and butylated hydroxytoluene and a combination thereof. In a preferred embodiment, the preservative is at a concentration from about 0.05% to about 0.2% w/v.

Electrolytes suitable for use in the present invention include, but are not limited to, magnesium ions, sodium chloride (“NaCl”), potassium chloride (“KCl”) and a combination thereof In a more preferred embodiment, the electrolyte is NaCl. In a more preferred embodiment, the concentration of the electrolyte is from about 0.01% to about 2.0% w/v, preferably about 0.9% w/v.

Buffers useful in the present invention include, but are not limited to, citrate buffer, borate buffer and phosphate buffer. Buffers may be present in compositions of the present invention at a concentration from about 1 millimolar (“mM”) to about 10 mM, preferably from about 2 mM to about 6 mM and more preferably from about 3 mM to about 4 mM.

Sorbate may be present in compositions of the present invention at a concentration from about 0.01% to about 0.5% w/v, preferably from about 0.05% to about 0.25% w/v and more preferably at about 0.1% w/v. In a preferred embodiment the sorbate is potassium sorbate.

As used herein, all numerical values relating to amounts, weights, and the like, that are defined as “about” each particular value is plus or minus 10%. For example, the phrase “about 5% w/v” is to be understood as “4.5% to 5.5% w/v.” Therefore, amounts within 10% of the claimed value are encompassed by the scope of the claims.

As used herein “% w/v” refers to the percent weight of the total composition.

As used herein the terms “subject” and “patient” are used interchangeably and refer, but are not limited to, a person or other animals.

As used herein, the term “prevent” or “preventing” refers to precluding, averting, obviating, forestalling, stopping, or hindering something from happening, especially by advance action.

As used herein, the term “treat” or “treating” refers to reversing, alleviating or slowing the progress of the disease, disorder, or condition to which such terms apply, or one or more symptoms of such disease, disorder, or condition.

As used herein, the term “administration” or “administering” refers to topical application, injection or administration via implants.

As used herein, the term “enhances the activity” refers to increasing the biological activity of the active ingredient, such as a glaucoma drug, for its intended purpose.

The articles “a,” “an” and “the” are intended to include the plural as well as the singular, unless the context clearly indicates otherwise.

### Ophthalmological Drugs

It is a discovery of the present invention that extremely low-dose brimonidine in combination with an ophthalmological drug is capable of greatly increasing residency time of the ophthalmological drug on the surface of an eye.

Extremely low-dose brimonidine (“ELDB”) further reduces systemic delivery of the ophthalmological drugs and thus reduces systemic side effects. This greater retention leads to greater intraocular absorption and thus reduces the amount and or frequency of administration, leading to further patient compliance.

Ophthalmological drugs that may benefit from co-administration with ELDB include, but are not limited to, those drugs that are indicated for the treatment of glaucoma, allergies, macular edema, macular degeneration, diabetic retinopathy, uveitis, retinitis, choroiditis and retinopathies associated with vascular leakage.

Ophthalmological drugs that may be present in compositions of the present invention or used in methods of the present invention include, but are not limited to:

glaucoma treatments including prostaglandins such as latanoprost, bimatoprost, travaprost, tafluprost and pharmaceutically acceptable salts thereof, rho kinase inhibitors such as netarsudil, beta-blockers such as carteolol, timoptic, timolol, betaloxol, levobunolol, metipranolol and pharmaceutically acceptable salts thereof, carbonic anhydrase inhibitors (“CAIs”) such as brinzolamide, dorzolamide, acetazolamide, methazolamide and pharmaceutically acceptable salts thereof, and latanoprostene bunod, lopidine and dexmedetomidine and pharmaceutically acceptable salts thereof and combinations thereof;

allergy treatments including naphazoline, antazoline, azelastine, carbinoxamine, cyproheptadine, emedastine, hydroxyzine, levocabastine, brompheniramine, chlorpheniramine, clemastine, diphenhydramine, ketotifen, loratadine, desloratadine, cetirizine, fexofenadine, olopatadine, acrivastine, ebastine, norastemizole, levocetirizine, mizolastine, pheniramine, pharmaceutically acceptable salts thereof and combinations thereof;

macular edema, macular degeneration and diabetic retinopathy treatments including ranibizumab, bevacizumab, pazopantinib, fluocinolone, axitinib, cabozantinib, foretinib, regorafenib, ponatinib, motesanib, cediranib, tivozanib, sorafenib, LY2457546, MGCD-265, MGCD-510, tivantinib, AMG458, JNJ-3887, EMD1214063, BMS794833, PHI1665752, SGX-523 and INCB280 and aflibercept;

uveitis treatments including steroidal anti-inflammatories such as prednisolone acetate, dexamethasone, bethamethasone, methylprednisolone, triamcinolone, prednisolone, prednisone, hydrocortisone and cortisone, immunosuppressants such as methotrexate, mycophenolate, azathioprine, and cyclosporine, biologic response modifiers such as adalimumab, infliximab, daclizumab, abatacept, and rituximab; and

non-steroidal anti-inflammatory (“NSAIDs”) including ketorolac, aspirin, celecoxib, diflunisal, etodolac, ibuprofen, indomethacin, ketoprofen, nabumetone, naproxen, oxaprozin, salsalate, sulindac, tolmetin and pharmaceutically acceptable salts thereof and combinations thereof.

Combination with Glaucoma Drugs

In one embodiment, the present invention is directed to an ophthalmological composition for the treatment of glaucoma comprising one or more active ingredients selected from the group consisting of netarsudil, a prostaglandin a prostaglandin analogue, a carbonic anhydrase inhibitor, a beta blocker and from about 0.005% to about 0.10% w/v brimonidine.

In one embodiment, the present invention is directed to an ophthalmological composition for the treatment of glaucoma comprising brimonidine at a concentration from about 0.01% to about 0.059% w/v and a second glaucoma drug.

In a preferred embodiment, the present invention is directed to an ophthalmological composition for the treatment of glaucoma comprising brimonidine at a concentration from about 0.01% to about 0.059% w/v, a second glaucoma drug, one or more nonionic surfactants and a viscosity enhancer.

In a preferred embodiment, the present invention is directed to an ophthalmological composition for the treatment of glaucoma comprising brimonidine at a concentration from about 0.01% to about 0.059% w/v, a second glaucoma drug, one or more nonionic surfactants, a viscosity enhancer, a buffer and a sorbate.

Glaucoma drugs other than brimonidine may be at concentrations from about 0.001% to about 5% w/v. Preferably: prostaglandins are at a concentration from about 0.001% to about 0.1% w/v, more preferably from about 0.0015% to about 0.03% w/v; beta-blockers are at a concentration from about 0.1% to about 1% w/v, more preferably from about 0.25% to about 0.5% w/v; rho kinase inhibitors are at a concentration from about 0.01% to about 1% w/v, preferably from about 0.01% to about 0.05% w/v, and CAIS are at a concentration from about 0.5% to about 5% w/v, more preferably from about 1% to about 2% w/v. Glaucoma drugs for ophthalmological administration are currently marketed under the following concentrations of active ingredients: 0.004% travaprost; 0.01% and 0.03% bimatoprost; 0.0015% tafluprost; 0.005% latanoprost; 0.02% netarsudil; 0.25% and 0.5% timolol maleate; 0.25% and 0.5% timolol hemihydrate; 0.25% and 0.5% betaxolol hydrochloride (“HCl”); 0.25% and 0.5% levobunolol HCl; 1% brinzolamide; and 2% dorzolamide.

Specific combinations of brimonidine and a second glaucoma drug include, but are not limited to: about 0.01% to about 0.059% w/v brimonidine and about 0.0015% to about 0.03% w/v latanoprost; about 0.035% to 0.050% w/v brimonidine and about 0.005% w/v latanoprost; about 0.059% w/v brimonidine and about 0.005% w/v latanoprost; about 0.01% to about 0.050% w/v brimonidine and 0.03% w/v latanoprost; about 0.050% w/v brimonidine and about 0.03% w/v latanoprost; about 0.035% to about 0.059% w/v brimonidine and about 0.050% w/v bimatoprost; about 0.035% to about 0.050% w/v brimonidine and about 0.5% w/v timolol; from about 0.005% to about 0.059% w/v brimonidine and netarsudil; 0.065% w/v brimonidine and 0.02% w/v netarsudil; 0.073% w/v brimonidine and 0.02% w/v netarsudil; and from about 0.035% to about 0.050% w/v brimonidine and about 2% w/v dorzolamide.

Compositions of the present invention may have a pH from about 5.0 to about 8.0, more preferably from about 6.0 to about 7.5, even more preferably from about 7.2 to about 7.7 for brimonidine glaucoma combination drugs and even more preferably from about 7.4 to about 7.7, and most preferably from about 7.4 to about 7.6.

In a preferred embodiment, the present invention is directed to a composition comprising: about 0.03% w/v latanoprost, about 0.050% w/v brimonidine, about 4.0% w/v polysorbate 80, about 1.2% w/v carboxymethyl cellulose at a weight wherein 2.0% w/v provides a viscosity of 3,500 centipoise, optionally, about 5 mM borate buffer and, optionally, about 0.12% w/v potassium sorbate, wherein, optionally, the composition has a pH from about 7.4 to about 7.7.

In a preferred embodiment, the present invention is directed to a composition comprising:
netarsudil, preferably at a concentration of about 0.02% w/v;
from about 0.005% to about 0.1% w/v brimonidine, preferably from about 0.005% to about 0.059% w/v, more preferably about 0.065% or about 0.073% w/v;
about 3.5% w/v polysorbate;
about 1.4% w/v CMC;
optionally, one or more preservatives selected from the group consisting of BAK, sorbate, EDTA, methyl paraben and a peroxide based preservative.

In a preferred embodiment, the present invention is directed to a composition comprising: about 0.001% w/v latanoprost;
about 0.050% w/v brimonidine;
about 2.5% w/v polysorbate;
about 1.2% w/v hydroxypropylmethyl cellulose (“HPMC”);
about 5 mM borate buffer; and
optionally, about 0.1% w/v sorbate,
wherein the composition has a pH of about 7.4.

In another preferred embodiment, the present invention is directed to a composition comprising:
about 0.005% w/v latanoprost;
about 0.059% w/v brimonidine;
about 4.0% w/v polysorbate;
about 0.75% w/v mannitol;
about 0.9% w/v sodium chloride;
about 0.1% w/v EDTA;
about 5 mM borate buffer; and
optionally, about 0.12% w/v potassium sorbate,
wherein the composition has a pH of about 7.4.

Methocell® was used as the source of HPMC (Methocell is a registered trademark of and available from Dow Corning).

In another preferred embodiment, the present invention is directed to a composition comprising:
about 0.05% w/v timolol;
about 0.050% w/v brimonidine;
about 2.5% w/v polysorbate;
about 1.2% w/v HPMC;
about 4 mM borate buffer; and
optionally, about 0.1% w/v sorbate,
wherein the composition has a pH of about 7.4.

Compositions of the present invention for the treatment of glaucoma may be administered topically to the eye, via intraocular injection or via intraocular implant, preferably administration occurs via topical application.

Combination with Histamine Antagonists

Currently available anti-histamines (i.e. histamine antagonists) are not capable of fully alleviating all allergy symptoms including particularly the eye redness associated with cytokine induced vasodilation. It is a discovery of the present invention that the use of extremely low dose brimonidine (“ELDB”) in combination with an anti-histamine leads to not only greater reduction of redness but greater alleviation of allergy symptoms such as conjunctival swelling than the use of the anti-histamine alone.

In one embodiment, the present invention is directed to an ophthalmological composition for the treatment of allergies comprising brimonidine at a concentration from about 0.005% to about 0.059% w/v and a histamine antagonist.

Histamine antagonist may be at concentrations from about 0.01% to about 1% w/v, preferably from about 0.025% to about 0.7% w/v. Histamine antagonists for nasal or ophthalmological administration are currently marketed under the following concentrations of active ingredients: 0.05% naphazoline HCl; combination of 0.05% naphazoline HCl and 0.5% antazoline phosphate; combination of 0.05% naphazoline HCl and 0.3% pheniramine maleate; combination of 0.02675% naphazoline hydrochloride and 0.315% pheniramine maleate; 0.1% and 0.15% azelastine; 0.05% emedastine; 0.05% levocabastine HCl; 0.025% ketotifen; and 0.1%, 0.2% and 0.7% olopatadine.

In a preferred embodiment, the present invention is directed to a composition comprising:
about 0.0035% w/v ketotifen fumarate;
about 0.035% w/v brimonidine;
about 2.5% w/v polysorbate;
from about 0.1% to about 1.2% w/v HPMC;
about 4 mM citrate buffer; and
optionally, about 0.1% w/v sorbate,
wherein the composition has a pH of 6.5.
   Combination with Non-Steroidal Anti-Inflammatory Drugs

In one embodiment, the present invention is directed to an ophthalmological composition for the reduction of inflammation comprising brimonidine at a concentration from about 0.005% to about 0.059% w/v and an NSAID.

NSAIDs may be at concentrations from about 0.01% to about 10% w/v, preferably from about 0.01% to about 1% w/v, more preferably from about 0.02% to about 0.50% w/v.

Compositions of the present invention for the reduction of inflammation may be administered topically to the eye or nasal cavity via drops or spray.

### Methods of the Invention

Eye disease that may be treated with compositions and methods of the present invention include but are not limited to,

A) Glaucoma including open-angle glaucoma, angle-closure glaucoma, normal-tension glaucoma, congenital glaucoma, secondary glaucoma, pigmentary glaucoma, psuedoexfoliative glaucoma, traumatic glaucoma, neovascular glaucoma, irido corneal endothelial syndrome and uveitic glaucoma;

B) Allergies including allergic rhinitis and or ocular allergies; allergic rhinitis is an allergic inflammation of the nasal airways with attendant symptoms, including, but not limited to, rhinorrhea, nasal obstruction, nasal itching, sneezing, ocular pruritis; ocular allergies are any allergic diseases of the eye, including, but not limited to, seasonal/perennial allergic conjunctivitis, vernal keratoconjunctivitis, giant papillary conjunctivitis, perennial allergic conjunctivitis and atopic keratoconjunctivitis;

C) Maculopathies/Retinal degenerations including non-exudative (dry) age-related macular degeneration (“AMD”), prophylactic treatment of severe dry AMD to prevent onset of wet AMD, exudative (wet) AMD, choroidal neovascularization, diabetic retinopathy, particularly prophylactically in the treatment of background diabetic retinopathy to prevent diabetic macular edema and or proliferative retinopathy, the treatment prophylactically of proliferative retinopathy to prevent vitreous hemorrhage, and particularly preferentially in the presence of proliferative retinopathy where conventional treatments (antibody anti-VEGF) may induce increased fibrovascular change with contraction along the retina and possible retinal detachment, acute macular neuroretinopathy, central serous chorioretinopathy, cystoids macular edema and macular edema;

D) Uveitis/Retinitis/Choroiditis including acute multifocal placoid pigment epitheliopathy, Behcet's disease, Birdshot retinochoroidopathy, infectious (syphilis, lime, tuberculosis, toxoplasmosis), intermediate uveitis (pars planitis), multifocal choroiditis, multiple evanescent white dot syndrome, ocular sarcoidosis, posterior scleritis, serpiginous choroiditis, subretinal fibrosis, uveitis syndrome, and Vogt-Koyanagi-Harada syndrome;

E) Vascular diseases/Exudative diseases including Coat's disease, parafoveal telangiectasis, papillophlebitis, frosted branch angitis, sickle cell retinopathy, other hemoglobinopathies, angioid streaks and familial exudative vitreoretinopathy;

F) Traumatic/surgical diseases including sympathetic ophthalmia, uveitic retinal disease, retinal detachment, trauma from laser photocoagulation or photodynamic therapy, hypoperfusion during surgery, radiation retinotherapy and bone marrow transplant retinopathy;

G) Proliferative disorders including proliferative vitreal retinotherapy, epiretinal membranes, proliferative diabetic retinopathy and retinopathy of prematurity (retrolental fibroplastic);

H) Infectious disorders including ocular histoplasmosis, ocular toxocariasis, presumed ocular histoplasmosis syndrome, endophthalmitis, toxoplasmosis, retinal diseases associated with HIV infection, choroidal disease associated with HIV infection, uveitic disease associated with HIV infection, viral retinitis, acute retinal necrosis, progressive outer retinal necrosis, fungal retinal diseases, ocular syphilis, ocular tuberculosis, diffuse unilateral subacute neuroretinitis and myiasis;

I) Genetic disorders including systemic disorders with associated retinal dystrophies, congenital stationary night blindness, cone dystrophies, fundus flavimaculatus, Best's disease, Pattern dystrophy of the retinal pigmented epithelium, X-linked retinoschisis, Sorsby's fundus dystrophy, benign concentric maculopathy, Bietti's crystalline dystrophy, psuedoxanthoma elasticum and Osler Weber syndrome;

J) Retinal tears/holes including retinal detachment, macular hole and giant retinal tear;

K) Tumors including retinal disease associated with tumors, solid tumors, tumor metastasis, benign tumors (e.g. hemangiomas, neurofibromas, trachomas, pyogenic granulomas), congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma and intraocular lymphoid tumors;

L) Neovascular ischemia including, anterior segment ischemia syndromes, corneal neovascularization including post corneal surgery such as post penetrating keratoplasty, herpetic keratitis and other ischemic or corneal inflammatory conditions; and

M) Other diseases that may be treated by compositions and methods of the present invention include cancers not limited to chronic myeloid leukemia (“CIVIL”), acute lymphocytic leukemia, non-small cell lung cancer, pancreatic cancer, gastrointestinal stromal tumors, hypereosinophilic syndrome, systemic mastocytosis, breast cancer with HER2/neu overexpression, chronic phase or accelerated Ph-positive CML, renal cell cancer, and hepatocellular carcinoma.

### EXAMPLES

### Example 1

### Prophetic Intraocular Pressure Reduction Method

A subject with normo-tensive baseline intraocular pressure (“TOP”; <21 mm Hg) was prophetically treated for 5 consecutive days with a single instillation of two drops per eye per day of a composition containing 0.035% brimonidine, 0.005% latanoprost, and a combination of 0.03% brimonidine and 0.005% latanoprost. A 1-week washout period was observed between each of the three treatment cycles. Instillation was performed at 8:30 AM, followed by 30 seconds of punctual occlusion with instillation on days 1, 3, and 5.

IOP was measured at one or more of 4 hrs, 8 hrs, 12 hrs, 24 hrs, 32 hrs and comfort and side effect profile were qualitatively assessed.

### Results

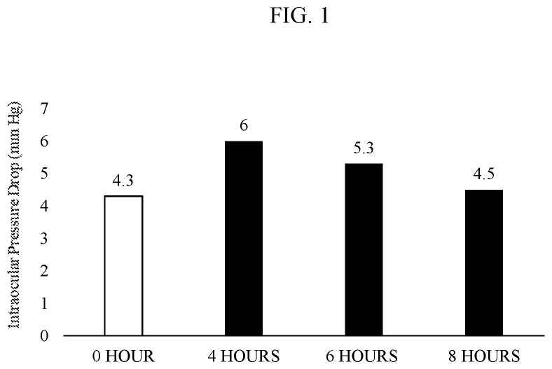

Based on preliminary data, the tested inventive formulations prophetically achieved greater IOP reduction than latanoprost alone. Further, a peak IOP reduction effect is prophetically achieved at about 4 to 8 hours after instillation and the IOP remained below the baseline 24 hours after instillation. FIG. 1 demonstrates a prophetic example of IOP reduction after administration of 0.005% latanoprost (0 hours, white bar), versus IOP reduction after administration of 0.03% brimonidine and 0.005% latanoprost (4, 6 and 8 hours, black bar). In fact, the reduction in IOP after administration of the combination of ELDB and latanoprost prophetically achieved synergy at all times and days except prior to instillation. Synergy will be calculated by taking the observed reduction in IOP and dividing by the expected reduction in IOP to give a “synergy factor”. Synergy factors greater than 1 demonstrate synergy. Expected IOP reduction will be calculated using the following formula where A is the IOP reduction for ELDB alone and B is the IOP reduction for latanoprost alone: A+B−(AB/100). Thus, the formulations of the invention prophetically demonstrate improved performance over full strength brimonidine and latanoprost alone under similar conditions of testing. Finally, during testing of latanoprost alone subject prophetically experienced ocular hyperemia that was not observed during the testing of brimonidine or the brimonidine and latanoprost combination demonstrating that extremely low dose brimonidine is capable of not only enhancing TOP reduction but also reducing attendant side effects of glaucoma drugs.

In conclusion, the combination of ELDB and a second glaucoma drug prophetically provide enhanced and synergistic relief of intraocular pressure that is either as good or better than that provided by the second glaucoma drug alone or the second glaucoma drug in combination with full strength brimonidine. Further, the combination of ELDB and the second glaucoma drug prophetically provide reduced or ameliorated side effects that normally occur when the second glaucoma drug is administered alone.

### Example 2

### Prophetic Allergy Relief

### Method

A subject suffering from nasal allergies was prophetically administered a combination of 0.005% to 0.050% w/v brimonidine and either 0.025% to 0.035% w/v ketotifen fumarate or 0.3% w/v pheniramine maleate for 7 consecutive days.

### Result

The tested inventive formulations prophetically achieved greater reduction in cytokines and inflammation than ketotifen or pheniramine alone. Further, the tested inventive formulations prophetically reduced eye redness associated with ketotifen and pheniramine. Finally, the tested inventive formulations prophetically whitened eyes of the subject beyond the baseline whiteness.

What is claimed is:

1. An ophthalmological composition comprising an ophthalmological drug and brimonidine at a concentration from about 0.005% to about 0.10% w/v, wherein w/v denotes weight by total volume of the composition.

2. The composition of claim 1, wherein brimonidine is at a concentration from about 0.01% to about 0.075% w/v.

3. The composition of claim 1, wherein brimonidine is at a concentration from about 0.06% to about 0.07% w/v.

4. The composition of claim 1, wherein brimonidine is at a concentration from about 0.025% to about 0.045% w/v.

5. The composition of claim 1, wherein brimonidine is at a concentration from about 0.025% to about 0.035% w/v.

6. The composition of claim 1, wherein the ophthalmological drug is selected from the group consisting of latanoprost, bimatoprost, travaprost, tafluprost, netarsudil, carteolol, timoptic, timolol, betaloxol, levobunolol, metipranolol, brinzolamide, dorzolamide, acetazolamide, methazolamide, latanoprostene bunod, lopidine, dexmedetomidinenaphazoline, antazoline, azelastine, carbinoxamine, cyproheptadine, emedastine, hydroxyzine, levocabastine, brompheniramine, chlorpheniramine, clemastine, diphenhydramine, ketotifen, loratadine, desloratadine, cetirizine, fexofenadine, olopatadine, acrivastine, ebastine, norastemizole, levocetirizine, mizolastine, pheniramine, fluocinolone, prednisolone, dexamethasone, bethamethasone, methylprednisolone, triamcinolone, prednisolone, prednisone, hydrocortisone, cortisone, methotrexate, mycophenolate, azathioprine, cyclosporine ketorolac, aspirin, celecoxib, diflunisal, etodolac, ibuprofen, indomethacin, ketoprofen, nabumetone, naproxen, oxaprozin, salsalate, sulindac, tolmetin and pharmaceutically acceptable salts thereof and ranibizumab, bevacizumab, pazopantinib, aflibercept, adalimumab, infliximab, daclizumab, abatacept, and rituximab and combinations thereof.

7. A method of treating an eye disease comprising administering a composition of claim 1 to a subject in need thereof.

8. The method of claim 7, wherein the eye disease is selected from the group consisting of glaucoma, allergies, macular edema, macular degeneration, diabetic retinopathy, uveitis, retinitis, choroiditis, retinopathies associated with vascular leakage and combinations thereof.

9. A method of treating an eye disease comprising topically administering an ophthalmological drug concurrently or sequentially with brimonidine at a concentration from about 0.005% to about 0.1% w/v to a subject in need thereof, wherein w/v denotes weight by total volume.

10. The method of claim 9, wherein brimonidine is at a concentration from about 0.005% to about 0.059% w/v and wherein brimonidine enhances the activity of the ophthalmological drug.

11. The method of claim 10, wherein brimonidine is at a concentration from about 0.06% to about 0.1% w/v and wherein aqueous humor production in the eye is reduced.

12. The method of claim 10, wherein brimonidine is formulated in a topical composition comprising brimonidine, one or more surfactants at a total concentration from about 1.0% to about 5.0% w/v and a viscosity enhancer.

13. The method of claim 12, wherein the viscosity enhancer is selected from the group consisting of a cellulose derivative, a carbomer, a gum and a hyaluronate.

14. The method of claim 13, wherein the viscosity enhancer is carboxymethyl cellulose at a concentration from about 0.75% to about 1.75% w/v.

15. The method of claim 9, wherein brimonidine is at a concentration from about 0.01% to about 0.059% w/v.

16. The method of claim 9, wherein brimonidine is at a concentration from about 0.025% to about 0.045% w/v.

17. The method of claim 9, wherein brimonidine is at a concentration from about 0.025% to about 0.035% w/v.

18. A method of increasing residency time of an ophthalmological drug on the surface of an eye comprising topically administering the ophthalmological drug concurrently or sequentially with brimonidine at a concentration from about 0.005% to about 0.059% w/v on the surface of the eye, wherein w/v denotes weight by total volume.

19. The method of claim 18, wherein brimonidine is at a concentration from about 0.01% to about 0.059% w/v.

20. The method of claim 18, wherein brimonidine is at a concentration from about 0.025% to about 0.045% w/v.

21. The method of claim 18, wherein brimonidine is at a concentration from about 0.025% to about 0.035% w/v.

22. An ophthalmological composition for the treatment of glaucoma comprising one or more active ingredients selected from the group consisting of netarsudil, a prostaglandin a prostaglandin analogue, a carbonic anhydrase inhibitor, a beta blocker and from about 0.005% to about 0.10% w/v brimonidine.

23. The composition of claim 22 further comprising 3.5% w/v polysorbate, 1.4% w/v carboxymethyl cellulose and wherein the one or more active ingredients are netarsudil and brimonidine.

24. The composition of claim 23, wherein the brimonidine is at a concentration from about 0.005% to about 0.059% w/v.

25. The composition of claim 23, wherein the brimonidine is at a concentration of about 0.065% w/v and the netarsudil is at a concentration of about 0.02% w/v.

26. The composition of claim 23, wherein the brimonidine is at a concentration of about 0.073% w/v and the netarsudil is at a concentration of about 0.02% w/v.
